(19) United States
(12) Patent Application Publication
(10) Pub. No.: US 2023/0129089 A1
(43) Pub. Date: Apr. 27, 2023

(54) PREPARATION OF A 1,3,5-TRIAZINYL BENZIMIDAZOLE

(71) Applicant: MEI Pharma, Inc., San Diego, CA (US)

(72) Inventor: David DUNCAN, San Diego, CA (US)

(21) Appl. No.: 17/915,284

(22) PCT Filed: Apr. 2, 2021
(86) PCT No.: PCT/US2021/025560

§ 371 Date: Sep. 28, 2022

Related U.S. Application Data
(60) Provisional application No. 63/006,564, filed on Apr. 7, 2020.

(51) Int. Cl. C07D 401/14; B01J 31/24

(52) U.S. Cl. CPC C07D 401/14; B01J 31/2409

(57) ABSTRACT

Described herein is the preparation of a 1,3,5-triazinyl benzimidazole and chemical intermediates used in the synthetic process.

### CROSS-REFERENCE

This International Patent Application claims the benefit of U.S. Provisional Patent Application No. 63/006,564, filed Apr. 7, 2020, which is incorporated herein by reference in its entirety.

### BACKGROUND OF THE INVENTION

Phosphoinositide-3-kinases (PI3Ks) are a group of lipid kinases, which phosphorylate the 3-hydroxyl of phosphoinositides. They are classified into at least three classes (classes I, II, and III) and play an important role in cellular signaling (Stephens et al., Curr. Opin. Pharmacol. 2005, 5, 357). Class I enzymes are further classified into classes Ia and Ib based on their mechanism of activation. Class Ia PI3Ks are heterodimeric structures consisting of a catalytic subunit (p110α, p110β, or p110δ) in complex with a regulatory p85 subunit, while class-Ib PI3K (p110γ) is structurally similar but lacks the p85 regulatory subunit, and instead is activated by βγ subunits of heterotrimeric G-proteins (Walker et al., Mol. Cell. 2000, 6, 909).

PI3Ks play a variety of roles in normal tissue physiology (Foukas & Shepherd, Biochem. Soc. Trans. 2004, 32, 330; Shepherd, Acta Physiol. Scand. 2005, 183, 3), with p110α having a specific role in cancer growth, p110β in thrombus formation mediated by integrin α₁₁ββ (Jackson et al., Nat. Med. 2005, 11, 507), and p110γ in inflammation, rheumatoid arthritis, and other chronic inflammation states (Barber et al., Nat. Med. 2005, 11, 933; Camps et al., Nat. Med. 2005, 11, 936; Rommel et al., Nat. Rev. 2007, 7, 191; and Ito, et al., J. Pharm. Exp. Therap. 2007, 321, 1). Therefore, there is a need for PI3K inhibitors, and methods for manufacturing them, for treating cancer and/or inflammatory diseases.

### SUMMARY OF THE INVENTION

In one aspect, described herein is a process for preparing Compound G:

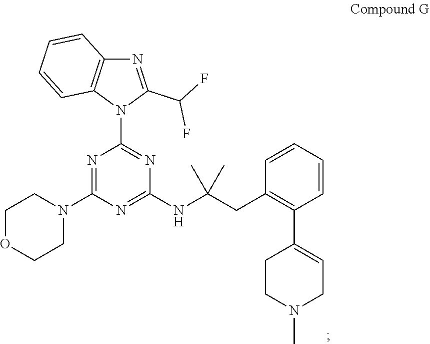 [SMILES: CN1CC=C(c2ccccc2CC(C)(C)Nc2nc(N3CCOCC3)nc(-n3c(C(F)F)nc4ccccc43)n2)CC1]

comprising contacting Compound E:

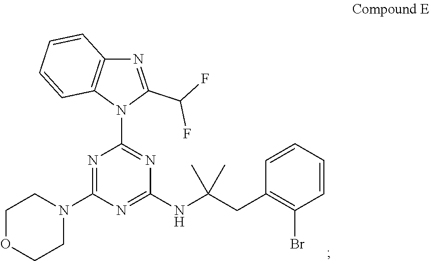 [SMILES: CC(C)(Cc1ccccc1Br)Nc1nc(N2CCOCC2)nc(-n2c(C(F)F)nc3ccccc32)n1]

with Compound F:

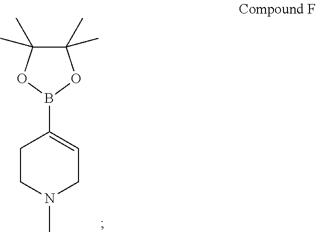

in the presence of a base, a catalyst, and a solvent.

In some embodiments, the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is potassium carbonate.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is tetrahydrofuran.

In some embodiments, the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃.CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂.CH₂Cl₂, Pd(dtbpf)Cl₂, Pd(MeCN)₄(BF₄)₂, PdCl₂, XPhos-Pd-G3, Pd-PEPPSI™-IPr, Pd-PEPPSI™-SIPr, and Pd-PEPPSI™-IPent. In some embodiments, the catalyst is Pd(dppf)Cl₂. In some embodiments, Compound E, Compound F, the base, the catalyst, and the solvent are stirred: for no longer than 45 hours; and at a temperature of between about 50° C. and about 60° C.

In some embodiments, the process comprises precipitating Compound G and isolating it by filtration.

In some embodiments, the process provides Compound G in a synthetic yield of greater than about 75%. In some embodiments, the process provides Compound G in a synthetic yield of greater than about 80%.

In some embodiments, the process further comprises contacting Compound G:

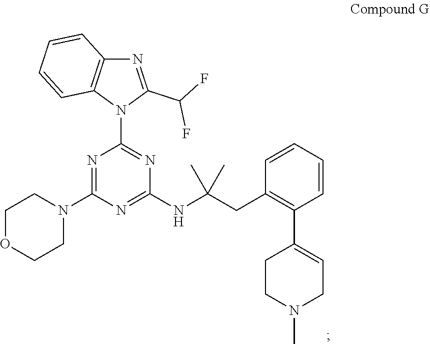 [SMILES: CN1CC=C(c2ccccc2CC(C)(C)Nc2nc(N3CCOCC3)nc(-n3c(C(F)F)nc4ccccc43)n2)CC1]

with gaseous hydrogen in the presence of a catalyst and a solvent to provide Compound 1:

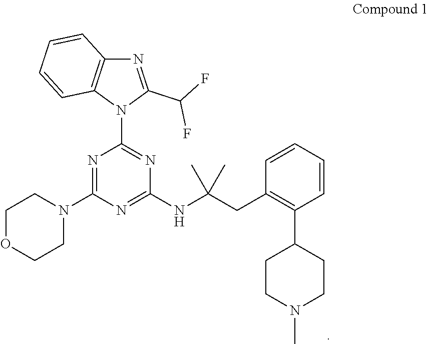 [SMILES: CN1CCC(c2ccccc2CC(C)(C)Nc2nc(N3CCOCC3)nc(-n3c(C(F)F)nc4ccccc43)n2)CC1]

In some embodiments, the catalyst is selected from Pd/C, Pd(OH)₂, Pd(OH)₂/C, Pd/Al₂O₃, Pd(OAc)₂/Et₃SiH, (PPh₃)₃RhCl, and PtO₂. In some embodiments, the catalyst is Pd(OH)₂/C.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is 1,4-dioxane.

In some embodiments, Compound G, the gaseous hydrogen, the catalyst, and the solvent are stirred: for no longer than 1 hour; and at a temperature of between about 45° C. and about 55° C.

In some embodiments, the process comprises precipitating Compound 1 and isolating it by filtration.

In some embodiments, the process provides Compound 1 in a synthetic yield of greater than about 60%.

In some embodiments, Compound E:

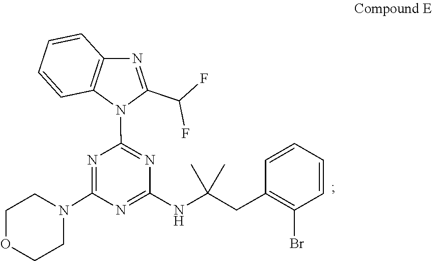 [SMILES: CC(C)(Cc1ccccc1Br)Nc1nc(N2CCOCC2)nc(-n2c(C(F)F)nc3ccccc32)n1]

is prepared by contacting Compound C:

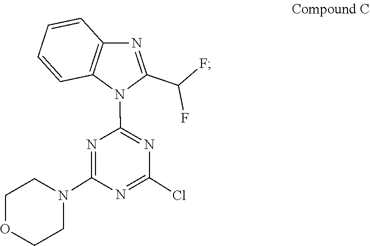 [SMILES: FC(F)c1nc2ccccc2n1-c1nc(Cl)nc(N2CCOCC2)n1]

with Compound D:

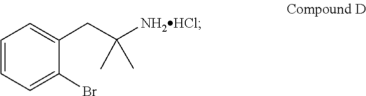

in the presence of a base and a solvent.

In some embodiments, the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is potassium carbonate.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is 1,4-dioxane.

In some embodiments, Compound C, Compound D, the base, and the solvent are stirred: for no longer than 40 hours; and at a temperature of between about 80° C. and about 90° C.

In some embodiments, the process comprises precipitating Compound E and isolating it by filtration.

In some embodiments, the process provides Compound E in a synthetic yield of greater than about 90%. In some embodiments, the process provides Compound E in a synthetic yield of greater than about 95%.

In some embodiments, Compound C:

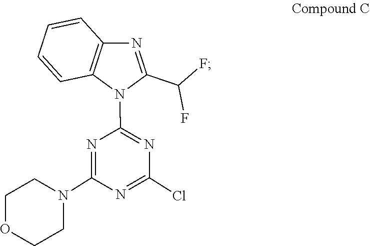 [SMILES: FC(F)c1nc2ccccc2n1-c1nc(Cl)nc(N2CCOCC2)n1]

is prepared by contacting Compound A:

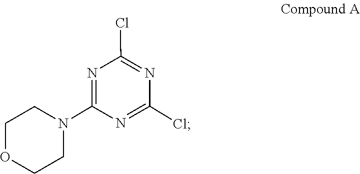 [SMILES: Clc1nc(Cl)nc(N2CCOCC2)n1]

with Compound B:

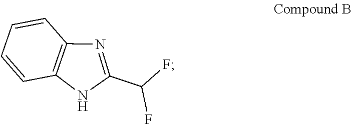 [SMILES: FC(F)c1nc2ccccc2[nH]1]

in the presence of a base and a solvent.

In some embodiments, the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is potassium carbonate.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is acetone.

In some embodiments, Compound A, Compound B, the base, and the solvent are stirred: for no longer than 18 hours; and at a temperature of between about 40° C. and about 50° C.

In some embodiments, the process comprises precipitating Compound C and isolating it by filtration.

In some embodiments, the process provides Compound C in a synthetic yield of greater than about 80%. In some embodiments, the process provides Compound C in a synthetic yield of greater than about 90%.

In another aspect, provided herein is a reaction mixture comprising

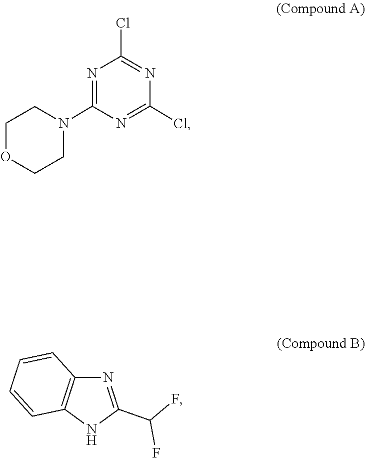 [SMILES: Clc1nc(Cl)nc(N2CCOCC2)n1 ; FC(F)c1nc2ccccc2[nH]1]

a base, and a solvent. In some embodiments, the base is potassium carbonate.

In some embodiments, the solvent is acetone.

In another aspect, provided herein is a reaction mixture comprising

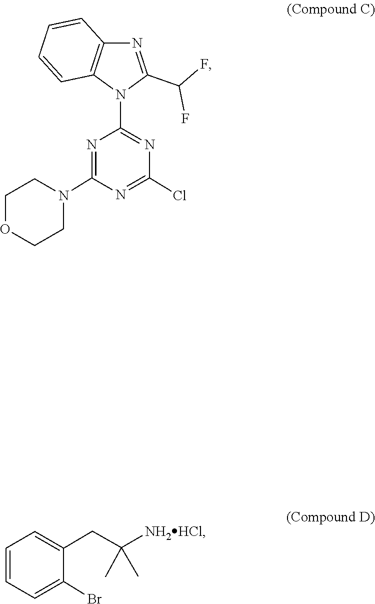

a base, and a solvent.

In some embodiments, the base is potassium carbonate.

In some embodiments, the solvent is 1,4-dioxane.

In another aspect, provided herein is a reaction mixture comprising

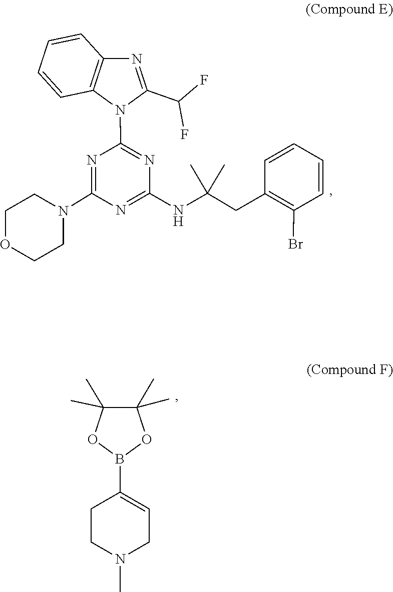

a base; a catalyst; and a solvent. In some embodiments, the base is potassium carbonate. In some embodiments, the catalyst is Pd(dppf)Cl₂. In some embodiments, the solvent is tetrahydrofuran.

In another aspect, provided herein is a reaction mixture comprising

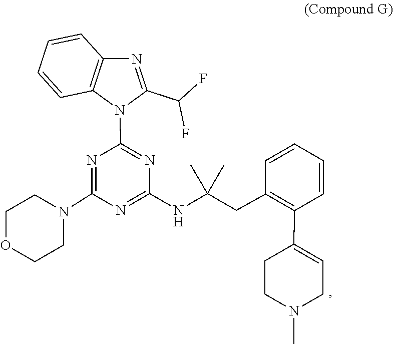 [SMILES: CN1CC=C(c2ccccc2CC(C)(C)Nc2nc(N3CCOCC3)nc(-n3c(C(F)F)nc4ccccc43)n2)CC1]

gaseous hydrogen; a catalyst; and a solvent. In some embodiments, the catalyst is Pd(OH)₂/C. In some embodiments, the solvent is 1,4-dioxane.

In another aspect, provided herein is a compound that is Compound G:

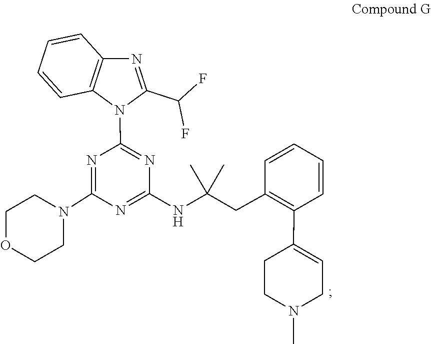 [SMILES: CN1CC=C(c2ccccc2CC(C)(C)Nc2nc(N3CCOCC3)nc(-n3c(C(F)F)nc4ccccc43)n2)CC1]

obtained by a process described herein.

In another aspect, provided herein is a compound that is Compound 1:

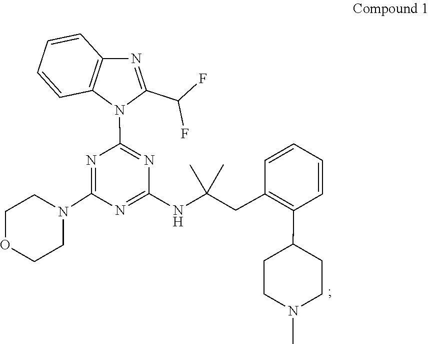 [SMILES: CN1CCC(c2ccccc2CC(C)(C)Nc2nc(N3CCOCC3)nc(-n3c(C(F)F)nc4ccccc43)n2)CC1]

obtained by a process described herein.

In another aspect, provided herein is a compound that is Compound E:

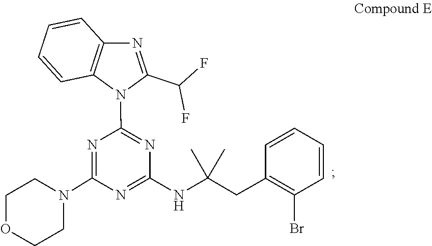 [SMILES: CC(C)(Cc1ccccc1Br)Nc1nc(N2CCOCC2)nc(-n2c(C(F)F)nc3ccccc32)n1]

obtained by a process described herein.

In another aspect, provided herein is a compound that is Compound C:

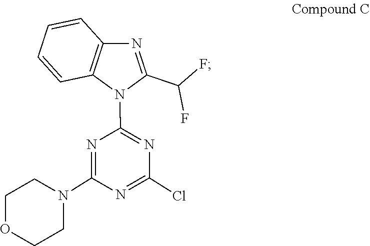 [SMILES: FC(F)c1nc2ccccc2n1-c1nc(Cl)nc(N2CCOCC2)n1]

obtained by a process described herein.

In another aspect, provided herein is a compound that is Compound G:

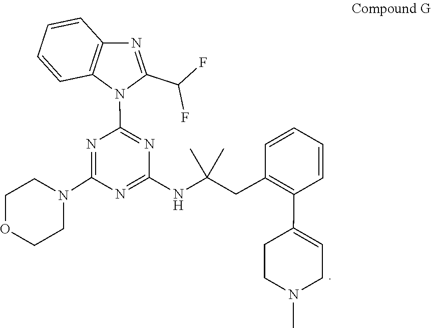 [SMILES: CN1CC=C(c2ccccc2CC(C)(C)Nc2nc(N3CCOCC3)nc(-n3c(C(F)F)nc4ccccc43)n2)CC1]

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Good manufacturing practices are needed for large scale manufacture of clinically useful drug candidates. Provided herein are certain processes and methods for the manufacture of 4-(2-(difluoromethyl)-1H-benzo[d]imidazole-1-yl)-N-(2-methyl-1-(2-(1-methylpiperidin-4-yl)phenyl)propan-2-yl)-6-morpholino-1,3,5-triazin-2-amine (Compound 1) or a pharmaceutically acceptable salt thereof.

### Definitions

As used in the specification and appended claims, unless specified to the contrary, the following terms have the meaning indicated below.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof.

When ranges are used herein for physical properties, such as molecular weight, or chemical properties, such as chemical formulae, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range varies between 1% and 15% of the stated number or numerical range.

The term “comprising” (and related terms such as “comprise” or “comprises” or “having” or “including”) is not intended to exclude that which in other certain embodiments, for example, an embodiment of any composition of matter, composition, method, or process, or the like, described herein, “consist of” or “consist essentially of” the described features.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, “treatment” or “treating” or “palliating” or “ameliorating” are used interchangeably herein. These terms refers to an approach for obtaining beneficial or desired results including but not limited to therapeutic benefit and/or a prophylactic benefit. Also, a therapeutic benefit is achieved with amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has been made.

### Improved Process for Preparation

Provided herein is a process useful for preparing a 1,3,5-triazinyl benzimidazole and its intermediates. In particular, provided herein is a process and method for the manufacture of 4-(2-(difluoromethyl)-1H-benzo[d]imidazole-1-yl)-N-(2-methyl-1-(2-(1-methylpiperidin-4-yl)phenyl)propan-2-yl)-6-morpholino-1,3,5-triazin-2-amine (Compound 1) or a pharmaceutically acceptable salt thereof, for example as shown in scheme 1.

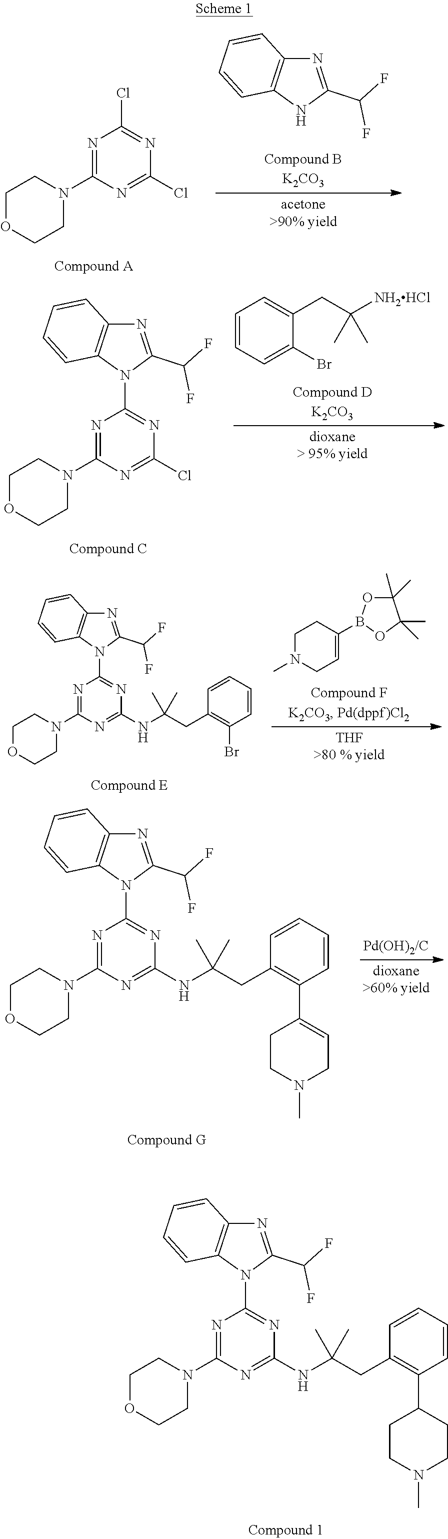

In some embodiments, the processes are improved over previously disclosed processes (e.g., as described in PCT/US2012/030640, shown in scheme 2). In some embodiments, the process described herein provides an increased overall yield.

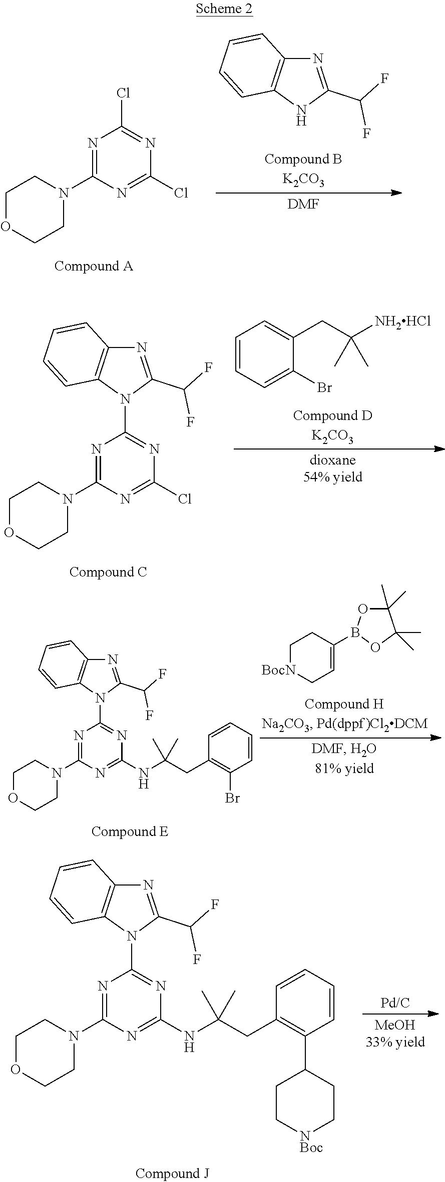

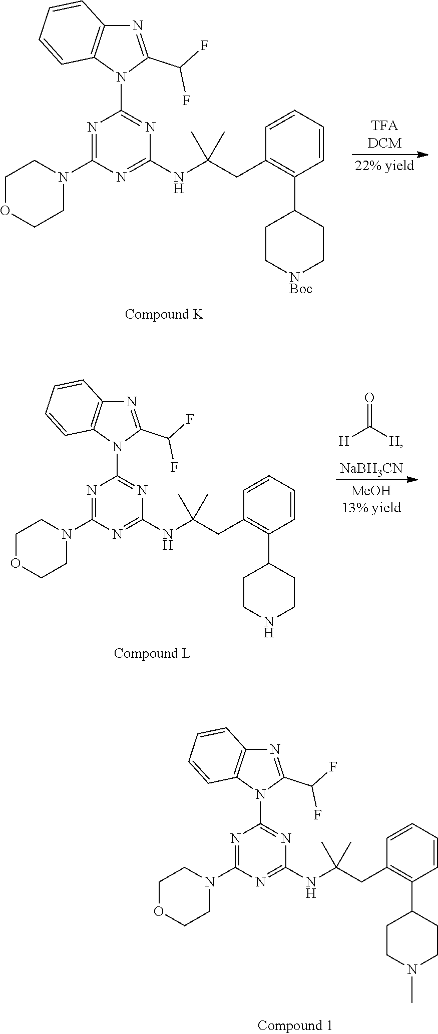

The process of the present disclosure provides Compound 1 from Compound A in four synthetic steps, as opposed to six synthetic steps for the process of PCT/US2012/030640. The lower number of overall steps results in lower usage of solvent and minimized waste and environmental impact. In particular, the process of the present disclosure avoids a number of solvents of concern, such as dichloromethane and dimethylformamide.

The process of the present disclosure provides Compound C in high yield and high purity. The present process is also operationally straightforward, as the reaction mixture displays improved stirring (e.g., no clumping) and is not sensitive to the particle size of K₂CO₃.

Furthermore, the process of the present disclosure avoids the use of highly undesirable reagents such as trifluoroacetic acid and formaldehyde, further minimizing the environmental impact of the process of the present disclosure. Additionally, the process of the present disclosure does not require column chromatography and hence avoids the use of silica gel.

The process of the present disclosure avoids the use of acid and dichloromethane, providing Compound G with increased purity and yield, and without the formation of side products resulting from benzimidazole hydrolysis or dichloromethane addition.

In some embodiments, the processes described herein provide Compound 1 in higher overall yields (e.g., 46% overall yield as compared with 2.9% overall yield for the process of PCT/US2012/030640). In some embodiments, the processes described herein provide Compound 1 in higher purity.

In one aspect, described herein is a process for preparing Compound G:

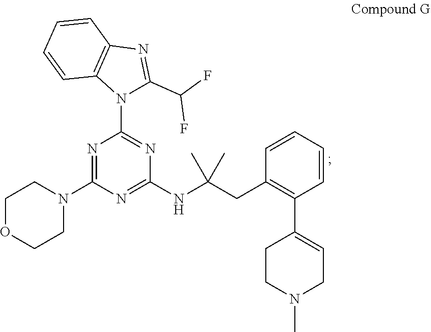 [SMILES: CN1CC=C(c2ccccc2CC(C)(C)Nc2nc(N3CCOCC3)nc(-n3c(C(F)F)nc4ccccc43)n2)CC1]

comprising contacting Compound E:

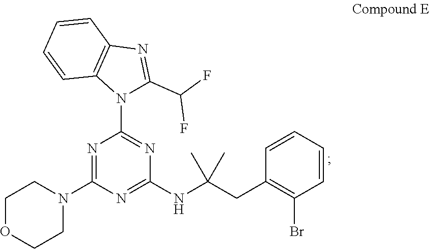 [SMILES: CC(C)(Cc1ccccc1Br)Nc1nc(N2CCOCC2)nc(-n2c(C(F)F)nc3ccccc32)n1]

with Compound F:

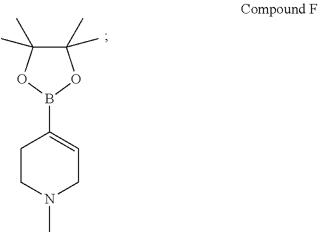

in the presence of a base, a catalyst, and a solvent.

In some embodiments, the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium carbonate. In some embodiments, the base is sodium carbonate. In some embodiments, the base is sodium bicarbonate. In some embodiments, the base is piperidine. In some embodiments, the base is 1,8-diazabicyclo[5.4.0]undec-7-ene. In some embodiments, the base is N,N-diisopropylethylamine. In some embodiments, the base is triethylamine.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃-CHCl₃, Pd(PPh₃)₄, Pd(OAc)₂, Pd(PCy₃)₂Cl₂, Pd(PPh₃)₂Cl₂, Pd[P(o-tol)₃]2Cl₂, Pd(amphos)Cl₂, Pd(dppf)Cl₂, Pd(dppf)Cl₂.CH₂Cl₂, Pd(dtbpf)Cl₂, Pd(MeCN)₄(BF₄)₂, PdCl₂, XPhos-Pd-G3, Pd-PEPPSI™-IPr, Pd-PEPPSI™-SIPr, and Pd-PEPPSI™-IPent. In some embodiments, the catalyst is Pd(acac)₂. In some embodiments, the catalyst is [Pd(allyl)Cl]₂. In some embodiments, the catalyst is Pd(MeCN)₂Cl₂. In some embodiments, the catalyst is Pd(dba)₂. In some embodiments, the catalyst is Pd(TFA)₂. In some embodiments, the catalyst is Pd₂(dba)₃. In some embodiments, the catalyst is Pd₂(dba)₃-CHCl₃. In some embodiments, the catalyst is Pd(PPh₃)₄. In some embodiments, the catalyst is Pd(OAc)₂. In some embodiments, the catalyst is Pd(PCy₃)₂Cl₂. In some embodiments, the catalyst is Pd(PPh₃)₂Cl₂. In some embodiments, the catalyst is Pd[P(o-tol)₃]₂Cl₂. In some embodiments, the catalyst is Pd(amphos)Cl₂. In some embodiments, the catalyst is Pd(dppf)Cl₂. In some embodiments, the catalyst is Pd(dppf)Cl₂.CH₂Cl₂. In some embodiments, the catalyst is Pd(dtbpf)Cl₂. In some embodiments, the catalyst is Pd(MeCN)₄(BF₄)₂. In some embodiments, the catalyst is PdCl₂. In some embodiments, the catalyst is XPhos-Pd-G3. In some embodiments, the catalyst is Pd-PEPPSI™-IPr. In some embodiments, the catalyst is Pd-PEPPSI™-SIPr. In some embodiments, the catalyst is Pd-PEPPSI™-IPent.

In some embodiments, Compound E, Compound F, the base, the catalyst, and the solvent are stirred: for no longer than 45 hours; and at a temperature of between about 50° C. and about 60° C.

In some embodiments, Compound E, Compound F, the base, the catalyst, and the solvent are stirred for no longer than 6 hours. In some embodiments, Compound E, Compound F, the base, the catalyst, and the solvent are stirred for no longer than 12 hours. In some embodiments, Compound E, Compound F, the base, the catalyst, and the solvent are stirred for no longer than 24 hours. In some embodiments, Compound E, Compound F, the base, the catalyst, and the solvent are stirred for no longer than 36 hours.

In some embodiments, Compound E, Compound F, the base, the catalyst, and the solvent are stirred at a temperature of about 50° C. In some embodiments, Compound E, Compound F, the base, the catalyst, and the solvent are stirred at a temperature of about 55° C. In some embodiments, Compound E, Compound F, the base, the catalyst, and the solvent are stirred at a temperature of about 60° C.

In some embodiments, the process comprises precipitating Compound G and isolating it by filtration.

In some embodiments, the process provides Compound G in a synthetic yield of greater than about 60%. In some embodiments, the process provides Compound G in a synthetic yield of greater than about 65%. In some embodiments, the process provides Compound G in a synthetic yield of greater than about 70%. In some embodiments, the process provides Compound G in a synthetic yield of greater than about 75%. In some embodiments, the process provides Compound G in a synthetic yield of greater than about 80%.

In some embodiments, the process further comprises contacting Compound G:

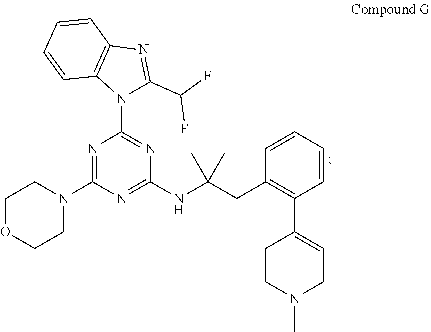 [SMILES: CN1CC=C(c2ccccc2CC(C)(C)Nc2nc(N3CCOCC3)nc(-n3c(C(F)F)nc4ccccc43)n2)CC1]

with gaseous hydrogen in the presence of a catalyst and a solvent to provide Compound 1:

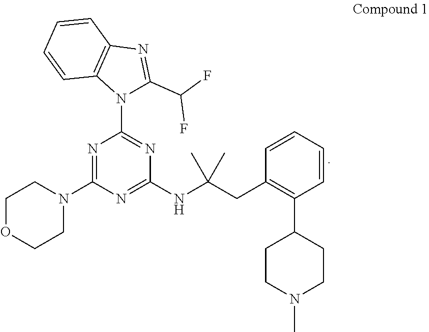 [SMILES: CN1CCC(c2ccccc2CC(C)(C)Nc2nc(N3CCOCC3)nc(-n3c(C(F)F)nc4ccccc43)n2)CC1]

In some embodiments, the catalyst is selected from Pd/C, Pd(OH)₂, Pd(OH)₂/C, Pd/Al₂O₃, Pd(OAc)₂/Et₃SiH, (PPh₃)₃RhCl, and PtO₂. In some embodiments, the catalyst is Pd/C. In some embodiments, the catalyst is Pd(OH)₂. In some embodiments, the catalyst is Pd(OH)₂/C. In some embodiments, the catalyst is Pd/Al₂O₃. In some embodiments, the catalyst is Pd(OAc)₂/Et₃SiH. In some embodiments, the catalyst is (PPh₃)₃RhCl. In some embodiments, the catalyst is PtO₂.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, Compound G, the gaseous hydrogen, the catalyst, and the solvent are stirred: for no longer than 1 hour; and at a temperature of between about 45° C. and about 55° C.

In some embodiments, Compound G, the gaseous hydrogen, the catalyst, and the solvent are stirred for no longer than 10 minutes. In some embodiments, Compound G, the gaseous hydrogen, the catalyst, and the solvent are stirred for no longer than 20 minutes. In some embodiments, Compound G, the gaseous hydrogen, the catalyst, and the solvent are stirred for no longer than 30 minutes. In some embodiments, Compound G, the gaseous hydrogen, the catalyst, and the solvent are stirred for no longer than 40 minutes. In some embodiments, Compound G, the gaseous hydrogen, the catalyst, and the solvent are stirred for no longer than 50 minutes. In some embodiments, Compound G, the gaseous hydrogen, the catalyst, and the solvent are stirred for no longer than 1 hour.

In some embodiments, Compound G, the gaseous hydrogen, the catalyst, and the solvent are stirred at a temperature of about 45° C. In some embodiments, Compound G, the gaseous hydrogen, the catalyst, and the solvent are stirred at a temperature of about 50° C. In some embodiments, Compound G, the gaseous hydrogen, the catalyst, and the solvent are stirred at a temperature of about 55° C.

In some embodiments, the process comprises precipitating Compound 1 and isolating it by filtration.

In some embodiments, the process provides Compound 1 in a synthetic yield of greater than about 50%. In some embodiments, the process provides Compound 1 in a synthetic yield of greater than about 55%. In some embodiments, the process provides Compound 1 in a synthetic yield of greater than about 60%.

In some embodiments, Compound E:

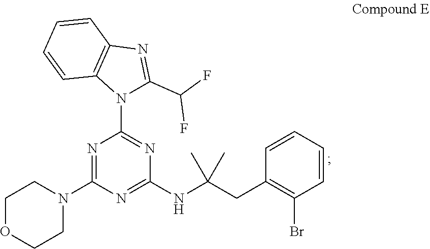 [SMILES: CC(C)(Cc1ccccc1Br)Nc1nc(N2CCOCC2)nc(-n2c(C(F)F)nc3ccccc32)n1]

is prepared by contacting Compound C:

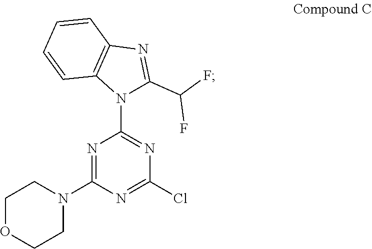 [SMILES: FC(F)c1nc2ccccc2n1-c1nc(Cl)nc(N2CCOCC2)n1]

with Compound D:

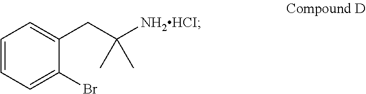

in the presence of a base and a solvent.

In some embodiments, the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium carbonate. In some embodiments, the base is sodium carbonate. In some embodiments, the base is sodium bicarbonate. In some embodiments, the base is piperidine. In some embodiments, the base is 1,8-diazabicyclo[5.4.0]undec-7-ene. In some embodiments, the base is N,N-diisopropylethylamine. In some embodiments, the base is triethylamine.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, Compound C, Compound D, the base, and the solvent are stirred: for no longer than 40 hours; and at a temperature of between about 80° C. and about 90° C.

In some embodiments, Compound C, Compound D, the base, the catalyst, and the solvent are stirred for no longer than 6 hours. In some embodiments, Compound C, Compound D, the base, the catalyst, and the solvent are stirred for no longer than 12 hours. In some embodiments, Compound C, Compound D, the base, the catalyst, and the solvent are stirred for no longer than 24 hours. In some embodiments, Compound C, Compound D, the base, the catalyst, and the solvent are stirred for no longer than 36 hours.

In some embodiments, Compound C, Compound D, the base, the catalyst, and the solvent are stirred at a temperature of about 80° C. In some embodiments, Compound C, Compound D, the base, the catalyst, and the solvent are stirred at a temperature of about 85° C. In some embodiments, Compound C, Compound D, the base, the catalyst, and the solvent are stirred at a temperature of about 90° C.

In some embodiments, the process comprises precipitating Compound E and isolating it by filtration.

In some embodiments, the process provides Compound E in a synthetic yield of greater than about 75%. In some embodiments, the process provides Compound E in a synthetic yield of greater than about 80%. In some embodiments, the process provides Compound E in a synthetic yield of greater than about 85%. In some embodiments, the process provides Compound E in a synthetic yield of greater than about 90%. In some embodiments, the process provides Compound E in a synthetic yield of greater than about 95%.

In some embodiments, Compound C:

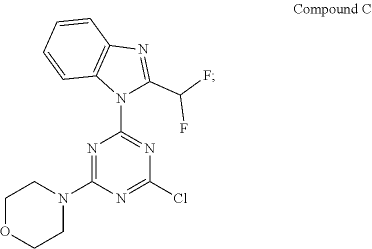 [SMILES: FC(F)c1nc2ccccc2n1-c1nc(Cl)nc(N2CCOCC2)n1]

is prepared by contacting Compound A:

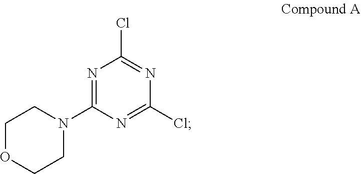 [SMILES: Clc1nc(Cl)nc(N2CCOCC2)n1]

with Compound B:

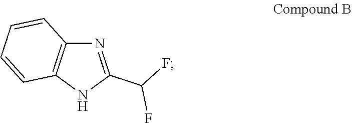 [SMILES: FC(F)c1nc2ccccc2[nH]1]

in the presence of a base and a solvent.

In some embodiments, the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium carbonate. In some embodiments, the base is sodium carbonate. In some embodiments, the base is sodium bicarbonate. In some embodiments, the base is piperidine. In some embodiments, the base is 1,8-diazabicyclo[5.4.0]undec-7-ene. In some embodiments, the base is N,N-diisopropylethylamine. In some embodiments, the base is triethylamine.

In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In some embodiments, Compound A, Compound B, the base, and the solvent are stirred: for no longer than 18 hours; and at a temperature of between about 40° C. and about 50° C.

In some embodiments, Compound A, Compound B, the base, and the solvent are stirred for no longer than 1 hour. In some embodiments, Compound A, Compound B, the base, and the solvent are stirred for no longer than 3 hours. In some embodiments, Compound A, Compound B, the base, and the solvent are stirred for no longer than 6 hours. In some embodiments, Compound A, Compound B, the base, and the solvent are stirred for no longer than 9 hours. In some embodiments, Compound A, Compound B, the base, and the solvent are stirred for no longer than 12 hours. In some embodiments, Compound A, Compound B, the base, and the solvent are stirred for no longer than 15 hours. In some embodiments, Compound A, Compound B, the base, and the solvent are stirred for no longer than 18 hours.

In some embodiments, Compound A, Compound B, the base, and the solvent are stirred at a temperature of about 40° C. In some embodiments, Compound A, Compound B, the base, and the solvent are stirred at a temperature of about 45° C. In some embodiments, Compound A, Compound B, the base, and the solvent are stirred at a temperature of about 50° C.

In some embodiments, the process comprises precipitating Compound C and isolating it by filtration.

In some embodiments, the process provides Compound C in a synthetic yield of greater than about 75%. In some embodiments, the process provides Compound C in a synthetic yield of greater than about 80%. In some embodiments, the process provides Compound C in a synthetic yield of greater than about 85%. In some embodiments, the process provides Compound C in a synthetic yield of greater than about 90%.

In another aspect, provided herein is a reaction mixture comprising

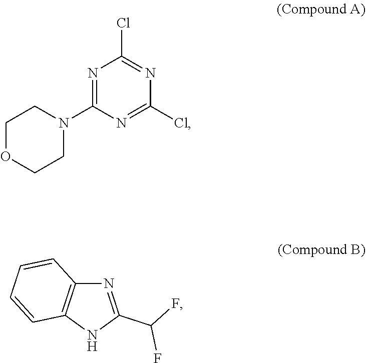 [SMILES: Clc1nc(Cl)nc(N2CCOCC2)n1 ; FC(F)c1nc2ccccc2[nH]1]

a base; and a solvent. In some embodiments, the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium carbonate. In some embodiments, the base is sodium carbonate. In some embodiments, the base is sodium bicarbonate. In some embodiments, the base is piperidine. In some embodiments, the base is 1,8-diazabicyclo[5.4.0]undec-7-ene. In some embodiments, the base is N,N-diisopropylethylamine. In some embodiments, the base is triethylamine. In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In another aspect, provided herein is a reaction mixture comprising

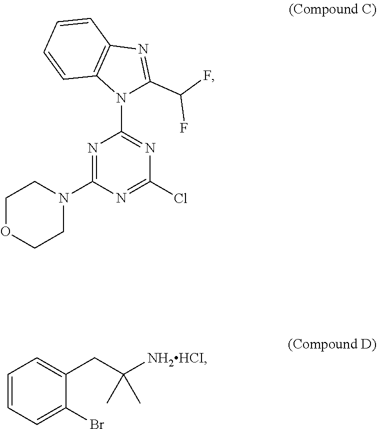

a base; and a solvent. In some embodiments, the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium carbonate. In some embodiments, the base is sodium carbonate. In some embodiments, the base is sodium bicarbonate. In some embodiments, the base is piperidine. In some embodiments, the base is 1,8-diazabicyclo[5.4.0]undec-7-ene. In some embodiments, the base is N,N-diisopropylethylamine. In some embodiments, the base is triethylamine. In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In another aspect, provided herein is a reaction mixture comprising

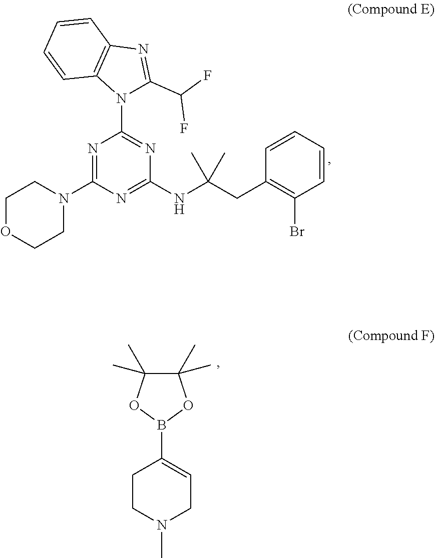

a base; a catalyst; and a solvent. In some embodiments, the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine. In some embodiments, the base is sodium hydroxide. In some embodiments, the base is potassium carbonate. In some embodiments, the base is sodium carbonate. In some embodiments, the base is sodium bicarbonate. In some embodiments, the base is piperidine. In some embodiments, the base is 1,8-diazabicyclo[5.4.0]undec-7-ene. In some embodiments, the base is N,N-diisopropylethylamine. In some embodiments, the base is triethylamine. In some embodiments, the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃-CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂.CH₂Cl₂, Pd(dtbpf)Cl₂, Pd(MeCN)₄(BF₄)₂, PdCl₂, XPhos-Pd-G3, Pd-PEPPSI™-IPr, Pd-PEPPSI™-SIPr, and Pd-PEPPSI™-IPent. In some embodiments, the catalyst is Pd(acac)₂. In some embodiments, the catalyst is [Pd(allyl)Cl]₂. In some embodiments, the catalyst is Pd(MeCN)₂Cl₂. In some embodiments, the catalyst is Pd(dba)₂. In some embodiments, the catalyst is Pd(TFA)₂. In some embodiments, the catalyst is Pd₂(dba)₃. In some embodiments, the catalyst is Pd₂(dba)₃-CHCl₃. In some embodiments, the catalyst is Pd(PPh₃)₄. In some embodiments, the catalyst is Pd(OAc)₂. In some embodiments, the catalyst is Pd(PCy₃)₂Cl₂. In some embodiments, the catalyst is Pd(PPh₃)₂Cl₂. In some embodiments, the catalyst is Pd[P(o-tol)₃]₂Cl₂. In some embodiments, the catalyst is Pd(amphos)Cl₂. In some embodiments, the catalyst is Pd(dppf)Cl₂. In some embodiments, the catalyst is Pd(dppf)Cl₂.CH₂Cl₂. In some embodiments, the catalyst is Pd(dtbpf)Cl₂. In some embodiments, the catalyst is Pd(MeCN)₄(BF₄)₂. In some embodiments, the catalyst is PdCl₂. In some embodiments, the catalyst is XPhos-Pd-G3. In some embodiments, the catalyst is Pd-PEPPSI™-IPr. In some embodiments, the catalyst is Pd-PEPPSI™-SIPr. In some embodiments, the catalyst is Pd-PEPPSI™-IPent. In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In another aspect, provided herein is a reaction mixture comprising

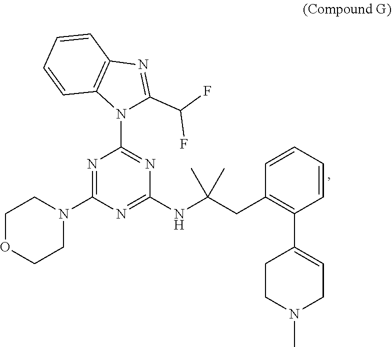 [SMILES: CN1CC=C(c2ccccc2CC(C)(C)Nc2nc(N3CCOCC3)nc(-n3c(C(F)F)nc4ccccc43)n2)CC1]

gaseous hydrogen; a catalyst; and a solvent.

In some embodiments, the catalyst is selected from Pd/C, Pd(OH)₂, Pd(OH)₂/C, Pd/Al₂O₃, Pd(OAc)₂/Et₃SiH, (PPh₃)₃RhCl, and PtO₂. In some embodiments, the catalyst is Pd/C. In some embodiments, the catalyst is Pd(OH)₂. In some embodiments, the catalyst is Pd(OH)₂/C. In some embodiments, the catalyst is Pd/Al₂O₃. In some embodiments, the catalyst is Pd(OAc)₂/Et₃SiH. In some embodiments, the catalyst is (PPh₃)₃RhCl. In some embodiments, the catalyst is PtO₂. In some embodiments, the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether. In some embodiments, the solvent is water. In some embodiments, the solvent is ethyl acetate. In some embodiments, the solvent is dichloromethane. In some embodiments, the solvent is tetrahydrofuran. In some embodiments, the solvent is diethyl ether. In some embodiments, the solvent is dimethylformamide. In some embodiments, the solvent is dimethylsulfoxide. In some embodiments, the solvent is methanol. In some embodiments, the solvent is ethanol. In some embodiments, the solvent is acetone. In some embodiments, the solvent is acetonitrile. In some embodiments, the solvent is 1,4-dioxane. In some embodiments, the solvent is hexane. In some embodiments, the solvent is methyl tert-butyl ether.

In another aspect, provided herein is a compound that is Compound G:

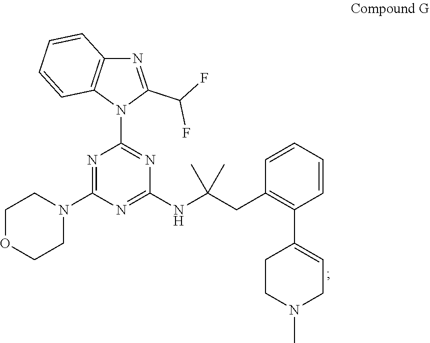 [SMILES: CN1CC=C(c2ccccc2CC(C)(C)Nc2nc(N3CCOCC3)nc(-n3c(C(F)F)nc4ccccc43)n2)CC1]

obtained by a process described herein.

In another aspect, provided herein is a compound that is Compound 1:

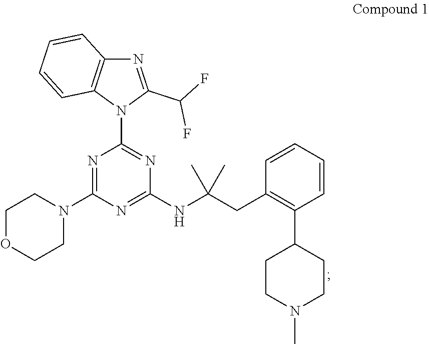 [SMILES: CN1CCC(c2ccccc2CC(C)(C)Nc2nc(N3CCOCC3)nc(-n3c(C(F)F)nc4ccccc43)n2)CC1]

obtained by a process described herein.

In another aspect, provided herein is a compound that is Compound E:

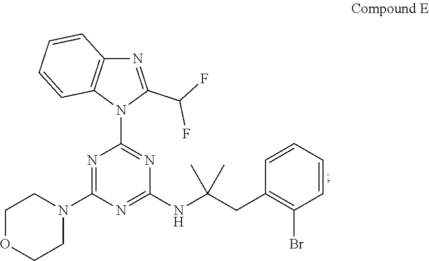 [SMILES: CC(C)(Cc1ccccc1Br)Nc1nc(N2CCOCC2)nc(-n2c(C(F)F)nc3ccccc32)n1]

obtained by a process described herein.

In another aspect, provided herein is a compound that is Compound C:

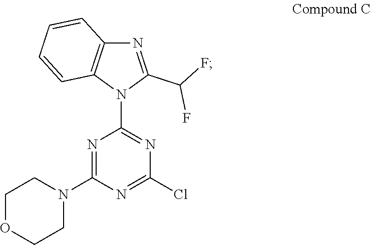 [SMILES: FC(F)c1nc2ccccc2n1-c1nc(Cl)nc(N2CCOCC2)n1]

obtained by a process described herein.

In another aspect, provided herein is a compound that is Compound G:

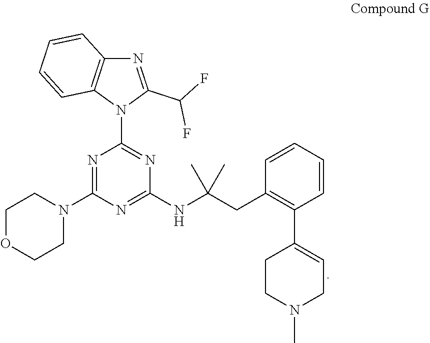 [SMILES: CN1CC=C(c2ccccc2CC(C)(C)Nc2nc(N3CCOCC3)nc(-n3c(C(F)F)nc4ccccc43)n2)CC1]

In some embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

### Compounds and Synthetic Intermediates

In some embodiments, the PI3K inhibitor described herein is 4-(2-(difluoromethyl)-1H-benzo[d]imidazole-1-yl)-N-(2-methyl-1-(2-(1-methylpiperidin-4-yl)phenyl)propan-2-yl)-6-morpholino-1,3,5-triazin-2-amine (Compound 1), or a pharmaceutically acceptable salt thereof:

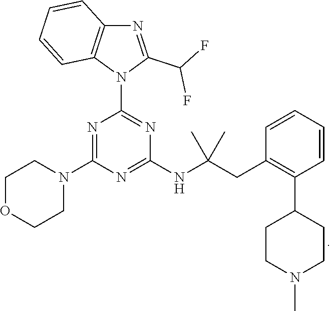 [SMILES: CN1CCC(c2ccccc2CC(C)(C)Nc2nc(N3CCOCC3)nc(-n3c(C(F)F)nc4ccccc43)n2)CC1]

In some embodiments, the starting materials for the synthesis of Compound 1 are

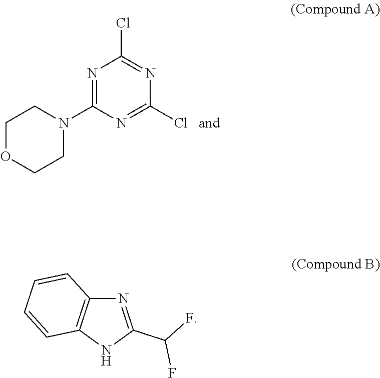 [SMILES: Clc1nc(Cl)nc(N2CCOCC2)n1 ; FC(F)c1nc2ccccc2[nH]1]

In some embodiments, an intermediate in the synthesis of Compound 1 is

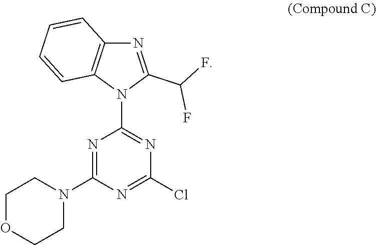 [SMILES: FC(F)c1nc2ccccc2n1-c1nc(Cl)nc(N2CCOCC2)n1]

In some embodiments, an intermediate in the synthesis of Compound 1 is

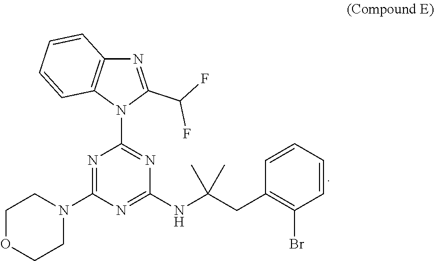 [SMILES: CC(C)(Cc1ccccc1Br)Nc1nc(N2CCOCC2)nc(-n2c(C(F)F)nc3ccccc32)n1]

In some embodiments, an intermediate in the synthesis of Compound 1 is

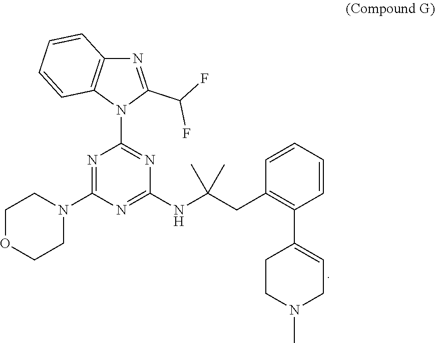 [SMILES: CN1CC=C(c2ccccc2CC(C)(C)Nc2nc(N3CCOCC3)nc(-n3c(C(F)F)nc4ccccc43)n2)CC1]

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

In some embodiments, the pharmaceutically acceptable salt of Compound 1 is an acetate, benzoate, besylate, bitartrate, carbonate, citrate, fumarate, gluconate, hydrobromide, hydrochloride, maleate, mesylate, nitrate, phosphate, salicylate, succinate, sulfate, or tartrate salt. In some embodiments, the pharmaceutically acceptable salt of Compound 1 is a mono-hydrochloride salt. In further embodiments, the pharmaceutically acceptable salt of Compound 1 is a mono-hydrochloride salt.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that are incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²p ³⁵S, ¹⁸F, and ³⁶Cl, respectively. Compounds described herein, and pharmaceutically acceptable salts, esters, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. Increased levels of deuterium incorporation produce a detectable kinetic isotope effect (KIE) that may affect the pharmacokinetic, pharmacologic and/or toxicologic parameters of Compound 1 in comparison to Compound 1 having naturally occurring levels of deuterium. In some embodiments, the isotopically labeled compound, or a pharmaceutically acceptable salt thereof, is prepared by any suitable method.

In some embodiments, at least one hydrogen in Compound 1 is replaced with deuterium.

In some embodiments, the compounds described herein are labeled by other means, including, but not limited to, the use of chromophores or fluorescent moieties, bioluminescent labels, or chemiluminescent labels.

### Methods of Treatment

The compounds described herein can be used in the preparation of medicaments for the modulation of PI3K, or for the treatment of diseases or conditions that would benefit, at least in part, from modulation of PI3K. In addition, a method for treating any of the diseases or conditions described herein in a subject in need of such treatment, involves administration of pharmaceutical compositions containing at least one compound described herein, or a pharmaceutically acceptable salt, or pharmaceutically acceptable solvate or hydrate thereof, in therapeutically effective amounts to said subject.

In another embodiments, provided herein is a method for treating, preventing, or ameliorating one or more symptoms of a proliferative disease in a subject, comprising administering to the subject a compound disclosed herein (e.g., Compound 1).

In certain embodiments, the proliferative disease is cancer. In certain embodiments, the proliferative disease is hematological cancer. In some embodiments, Compound 1 is used for treating chronic lymphocytic leukemia (CLL), small lymphocytic lymphoma (SLL), follicular lymphoma (FL), marginal zone B cell lymphoma, diffuse large B-cell lymphoma (DLBCL), high grade non-Hodgkin's lymphoma, mantle cell lymphoma (MCL). In certain embodiments, the proliferative disease is an inflammatory disease. In certain embodiments, the proliferative disease is an immune disorder.

### EXAMPLES

All chemicals, reagents, and solvents were purchased from commercial sources when available and used without further purification.

### Example 1: Synthesis of 4-(2-(difluoromethyl)-1H-benzo[d]imidazole-1-yl)-N-(2-methyl-1-(2-(1-methylpiperidin-4-yl)phenyl)propan-2-yl)-6-morpholino-1,3,5-triazin-2-amine (Compound 1)

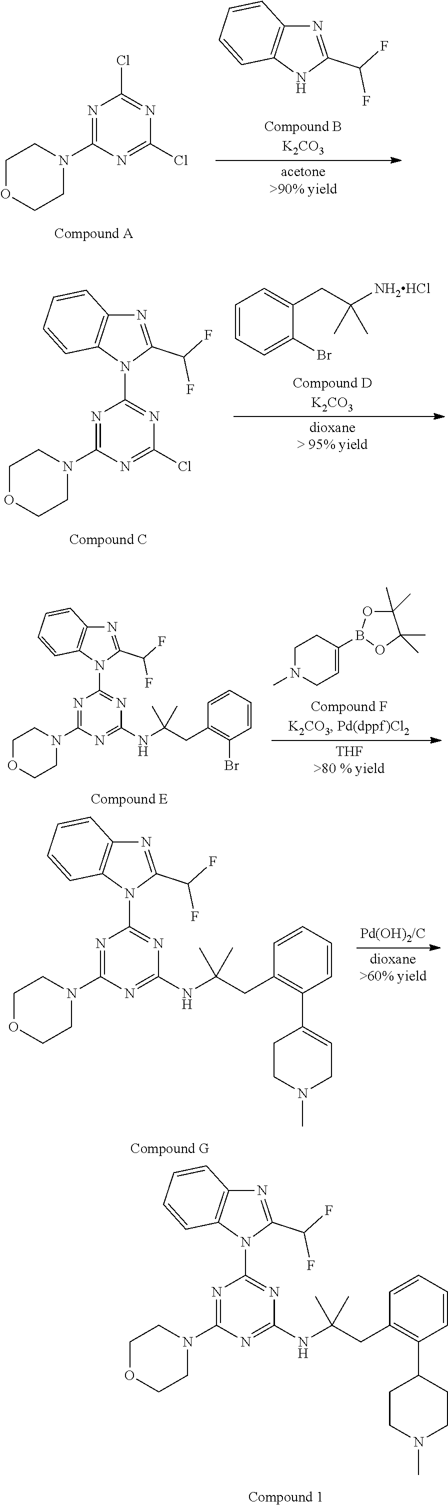

### Step 1: Preparation of 4-(4-chloro-6-(2-(difluoromethyl)-1H-benzo[d]imidazole-1-yl)-1,3,5-triazin-2-yl)morpholine (Compound C)

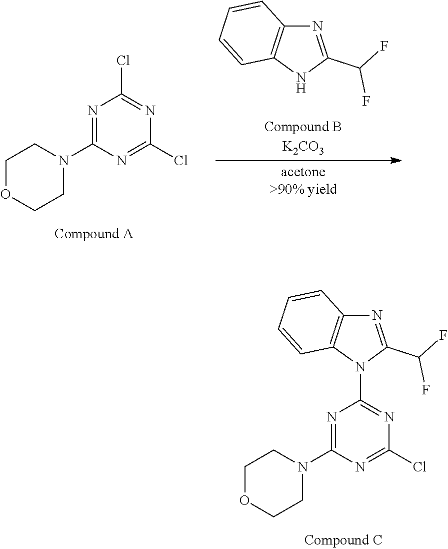 [SMILES: Clc1nc(Cl)nc(N2CCOCC2)n1 ; FC(F)c1nc2ccccc2[nH]1 ; FC(F)c1nc2ccccc2n1-c1nc(Cl)nc(N2CCOCC2)n1]

4-(4,6-dichloro-1,3,5-triazin-2-yl)morpholine (Compound A, 22.6 kg, 1 equiv.) and 2-(difluoromethyl)-1H-benzo[d]imidazole (Compound B, 16 kg, 1 equiv.) are coupled in acetone (250 kg, 0.3 M) and aqueous potassium carbonate (K₂CO₃, 26.6 kg). The slurry is heated to a temperature of 40 to 50° C. and stirred for no longer than 18 hours. Upon reaction completion, the slurry is cooled to a temperature of 15 to 25° C. and treated with water (575 kg). The resulting slurry is stirred at a temperature of 15 to 25° C. for no longer than 2 hours. The solid is isolated by filtration, washed with water and acetone, and dried under vacuum to afford 4-(4-chloro-6-(2-(difluoromethyl)-1H-benzo[d]imidazole-1-yl)-1,3,5-triazin-2-yl)morpholine (Compound C) in greater than 90% yield.

### Step 2: Preparation of N-(1-(2-bromophenyl)-2-methylpropan-2-yl)-4-(2-(difluoromethyl)-1H-benzo[d]imidazole-1-yl)-6-morpholino-1,3,5-triazin-2-amine (Compound E)

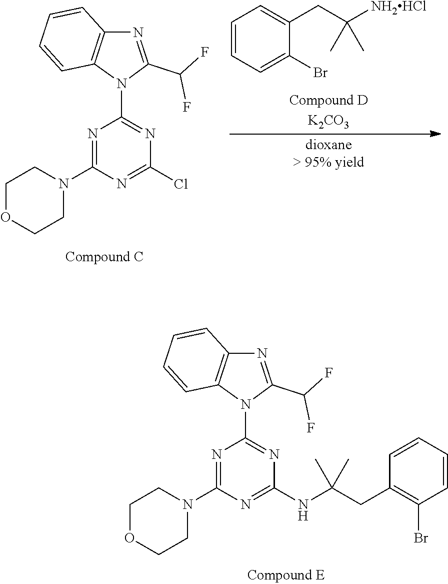

4-(4-chloro-6-(2-(difluoromethyl)-1H-benzo[d]imidazole-1-yl)-1,3,5-triazin-2-yl)morpholine (Compound C, 31.8 kg, 1 equiv.) and 1-(2-bromophenyl)-2-methylpropan-2-amine hydrochloride (Compound D, 23 kg, 1 equiv.) are coupled in 1,4-dioxane (470 kg, 0.2 M) and aqueous potassium carbonate (K₂CO₃, 82 kg). The resulting mixture is heated to a temperature of 80 to 90° C. and stirred for no longer than 40 hours. Upon reaction completion, the reaction mixture is cooled to a temperature of 40 to 50° C., and the aqueous phase is separated and discarded. The organic phase is washed with 28% aqueous potassium carbonate (K₂CO₃) at a temperature of 40 to 50° C. before being cooled to a temperature of 15 to 25° C. and treated with water. The resulting slurry is stirred at a temperature of 15 to 25° C. for no longer than 2 hours. The solid is isolated by filtration, washed with methyl tert-butyl ether, and dried under vacuum to afford N-(1-(2-bromophenyl)-2-methylpropan-2-yl)-4-(2-(difluoromethyl)-1H-benzo[d]imidazole-1-yl)-6-morpholino-1,3,5-triazin-2-amine (Compound E) in greater than 95% yield.

### Step 3: Preparation of 4-(2-(difluoromethyl)-1H-benzo[d]imidazole-1-yl)-N-(2-methyl-1-(2-(1-methylpiperidin-4-yl)phenyl)propan-2-yl)-6-morpholino-1,3,5-triazin-2-amine (Compound G)

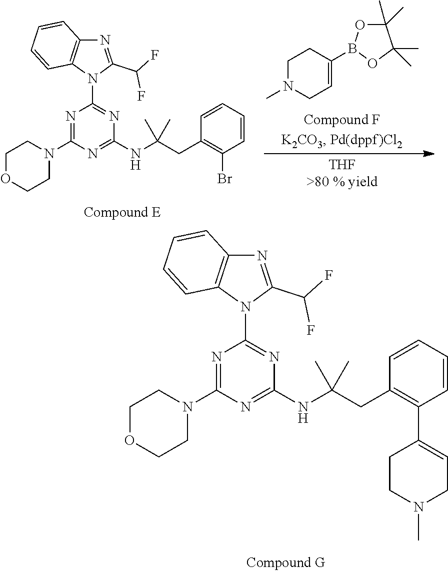

N-(1-(2-bromophenyl)-2-methylpropan-2-yl)-4-(2-(difluoromethyl)-1H-benzo[d]imidazole-1-yl)-6-morpholino-1,3,5-triazin-2-amine (Compound E, 44.6 kg, 1 equiv.) and 1-methyl-1,2,3,6-tetrahydropyridine-4-boronic acid pinacol ester (Compound F, 21.1 kg, 1.18 equiv.) are coupled with a catalytic amount of Pd(dppf)Cl₂ (1.5 kg, 0.026 equiv.) in THE (400 kg, 0.18 M) sparged with subsurface nitrogen for no longer than 1 hour) and aqueous potassium carbonate (K₂CO₃, 38.7 kg). The reaction mixture is heated to a temperature of 50 to 60° C. and stirred for no longer than 45 hours. Upon reaction completion, the reaction mixture is cooled to a temperature of 40 to 50° C. and passed through a polishing filter, and the aqueous phase is separated and discarded. The organic phase is treated with 20% aqueous potassium carbonate (K₂CO₃) with stirring at a temperature of 40 to 50° C. for no longer than 15 minutes. The reaction mixture is passed through a polishing filter and the aqueous phase is separated and discarded. The organic phase is partially concentrated until precipitation commences. Ethanol is added and the resulting slurry is heated at a temperature of 70 to 80° C. for no longer than 1 hour. The mixture is cooled to 15 to 25° C. and the solid is isolated by filtration, washed sequentially with ethanol, water, and ethanol, and dried under vacuum to afford 4-(2-(difluoromethyl)-1H-benzo[d]imidazole-1-yl)-N-(2-methyl-1-(2-(1-methylpiperidin-4-yl)phenyl)propan-2-yl)-6-morpholino-1,3,5-triazin-2-amine (Compound G) in greater than 80% yield.

### Step 4: Preparation of 4-(2-(difluoromethyl)-1H-benzo[d]imidazole-1-yl)-N-(2-methyl-1-(2-(1-methylpiperidin-4-yl)phenyl)propan-2-yl)-6-morpholino-1,3,5-triazin-2-amine (Compound 1)

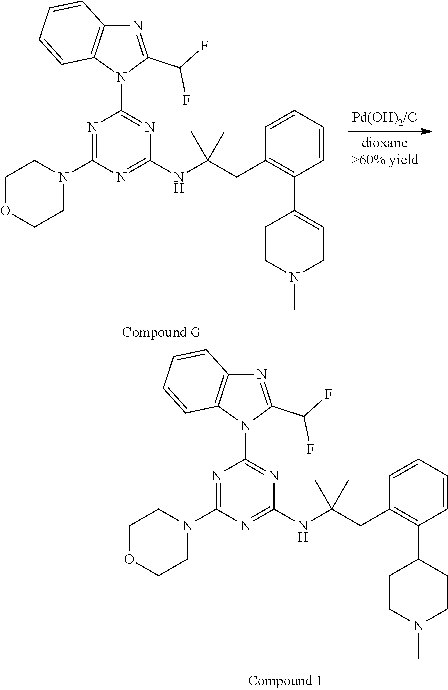 [SMILES: CN1CC=C(c2ccccc2CC(C)(C)Nc2nc(N3CCOCC3)nc(-n3c(C(F)F)nc4ccccc43)n2)CC1 ; CN1CCC(c2ccccc2CC(C)(C)Nc2nc(N3CCOCC3)nc(-n3c(C(F)F)nc4ccccc43)n2)CC1]

A slurry of 4-(2-(difluoromethyl)-1H-benzo[d]imidazole-1-yl)-N-(2-methyl-1-(2-(1-methylpiperidin-4-yl)phenyl)propan-2-yl)-6-morpholino-1,3,5-triazin-2-amine (Compound G, 37.2 kg, 1 equiv.) and 1,4-dioxane (778 kg, 0.09 M) is added to palladium hydroxide on activated carbon (Pd(OH)₂/C, 3.7 kg, 0.04 equiv.). The reaction mixture is purged with nitrogen several times, heated to a temperature of 45 to 55° C., and stirred for no longer than 1 hour. The nitrogen is vented and the reactor is charged with hydrogen gas (50 psi). Upon reaction completion, the reaction mixture is cooled to a temperature of 15 to 25° C., and the hydrogen is simultaneously vented. The reaction mixture is filtered through Celite® and the solvent is partially concentrated. The temperature is adjusted to 50 to 60° C. and water is added to complete precipitation. The resulting slurry is cooled to a temperature of 15 to 25° C. and stirred for no longer than 12 hours. The solid is collected by filtration, washed sequentially with 1,4-dioxane/water and ethanol, redissolved in tetrahydrofuran, and passed through activated carbon cartridges. The tetrahydrofuran is exchanged with ethanol via vacuum distillation. The resulting precipitate is treated with additional ethanol and heated to reflux at 78° C. Upon complete dissolution of the solid, the solvent is distilled at atmospheric pressure to approximately half volume. The resulting slurry is cooled to a temperature of 15 to 25° C. and stirred for no longer than 12 hours. The solid is collected by filtration, washed with ethanol, and dried at a temperature of 40 to 50° C. to afford 4-(2-(difluoromethyl)-1H-benzo[d]imidazole-1-yl)-N-(2-methyl-1-(2-(1-methylpiperidin-4-yl)phenyl)propan-2-yl)-6-morpholino-1,3,5-triazin-2-amine (Compound 1) in greater than 60% yield.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A process for preparing Compound G: 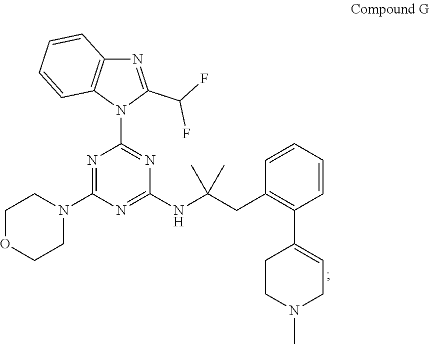 [SMILES: CN1CC=C(c2ccccc2CC(C)(C)Nc2nc(N3CCOCC3)nc(-n3c(C(F)F)nc4ccccc43)n2)CC1]
comprising contacting Compound E: 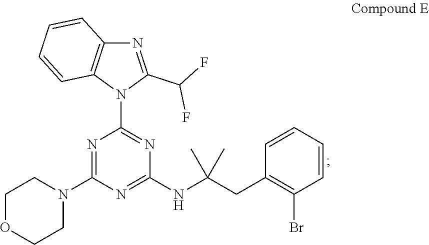 [SMILES: CC(C)(Cc1ccccc1Br)Nc1nc(N2CCOCC2)nc(-n2c(C(F)F)nc3ccccc32)n1]
with Compound F: 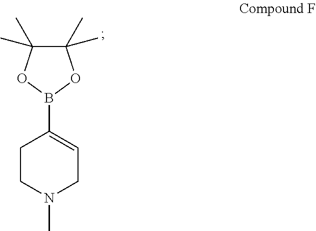
in the presence of a base, a catalyst, and a solvent.

2. The process of claim 1, wherein the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine.

3. The process of claim 1 or 2, wherein the base is potassium carbonate.

4. The process of any one of claims 1-3, wherein the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether.

5. The process of any one of claims 1-4, wherein the solvent is tetrahydrofuran.

6. The process of any one of claims 1-5, wherein the catalyst is selected from Pd(acac)₂, [Pd(allyl)Cl]₂, Pd(MeCN)₂Cl₂, Pd(dba)₂, Pd(TFA)₂, Pd₂(dba)₃, Pd₂(dba)₃-CHCl₃, Pd(PPh₃)₄, Pd(OAc)₂, Pd(PCy₃)₂Cl₂, Pd(PPh₃)₂Cl₂, Pd[P(o-tol)₃]₂Cl₂, Pd(amphos)Cl₂, Pd(dppf)Cl₂, Pd(dppf)Cl₂.CH₂Cl₂, Pd(dtbpf)Cl₂, Pd(MeCN)₄(BF₄)₂, PdCl₂, XPhos-Pd-G3, Pd-PEPPSI™-IPr, Pd-PEPPSI™-SIPr, and Pd-PEPPSI™-IPent.

7. The process of any one of claims 1-6, wherein the catalyst is Pd(dppf)Cl₂.

8. The process of any one of claims 1-7, wherein Compound E, Compound F, the base, the catalyst, and the solvent are stirred:
for no longer than 45 hours; and
at a temperature of between about 50° C. and about 60° C.

9. The process of any one of claims 1-8, comprising precipitating Compound G and isolating it by filtration.

10. The process of any one of claims 1-9, wherein the process provides Compound G in a synthetic yield of greater than about 75%.

11. The process of any one of claims 1-10, wherein the process provides Compound G in a synthetic yield of greater than about 80%.

12. The process of any one of claims 1-11, further comprising contacting Compound G: 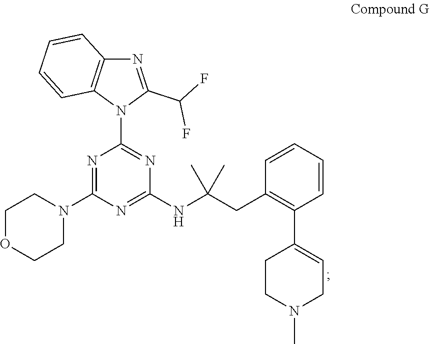 [SMILES: CN1CC=C(c2ccccc2CC(C)(C)Nc2nc(N3CCOCC3)nc(-n3c(C(F)F)nc4ccccc43)n2)CC1]
with gaseous hydrogen in the presence of a catalyst and a solvent to provide Compound 1:

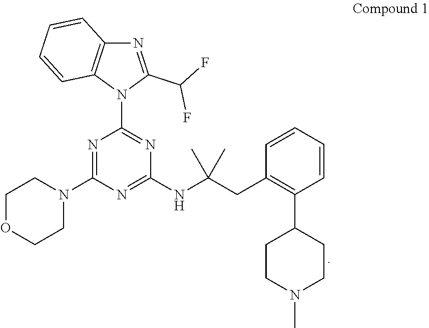 [SMILES: CN1CCC(c2ccccc2CC(C)(C)Nc2nc(N3CCOCC3)nc(-n3c(C(F)F)nc4ccccc43)n2)CC1]

13. The process of claim 12, wherein the catalyst is selected from Pd/C, Pd(OH)₂, Pd(OH)₂/C, Pd/Al₂O₃, Pd(OAc)₂/Et₃SiH, (PPh₃)₃RhCl, and PtO₂.

14. The process of claim 12 or 13, wherein the catalyst is Pd(OH)₂/C.

15. The process of any one of claims 12-14, wherein the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether.

16. The process of any one of claims 12-15, wherein the solvent is 1,4-dioxane.

17. The process of any one of claims 12-16, wherein the Compound G, the gaseous hydrogen, the catalyst, and the solvent are stirred:
for no longer than 1 hour; and
at a temperature of between about 45° C. and about 55° C.

18. The process of any one of claims 12-17, comprising precipitating Compound 1 and isolating it by filtration.

19. The process of any one of claims 12-18, wherein the process provides Compound 1 in a synthetic yield of greater than about 60%.

20. The process of any one of claims 1-19, wherein Compound E: 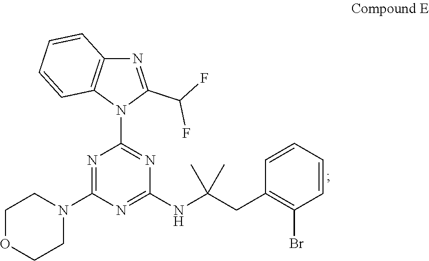 [SMILES: CC(C)(Cc1ccccc1Br)Nc1nc(N2CCOCC2)nc(-n2c(C(F)F)nc3ccccc32)n1]
is prepared by contacting Compound C: 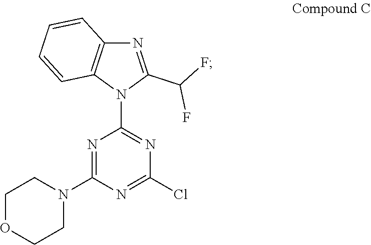 [SMILES: FC(F)c1nc2ccccc2n1-c1nc(Cl)nc(N2CCOCC2)n1]
with Compound D: 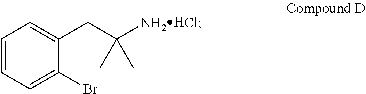
in the presence of a base and a solvent.

21. The process of claim 20, wherein the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine.

22. The process of claim 20 or 21, wherein the base is potassium carbonate.

23. The process of any one of claims 20-22, wherein the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether.

24. The process of any one of claims 20-23, wherein the solvent is 1,4-dioxane.

25. The process of any one of claims 20-24, wherein Compound C, Compound D, the base, and the solvent are stirred:
for no longer than 40 hours; and
at a temperature of between about 80° C. and about 90° C.

26. The process of any one of claims 20-25, comprising precipitating Compound E and isolating it by filtration.

27. The process of any one of claims 20-26, wherein the process provides Compound E in a synthetic yield of greater than about 90%.

28. The process of any one of claims 20-27, wherein the process provides Compound E in a synthetic yield of greater than about 95%.

29. The process of any one of claims 20-28, wherein Compound C: 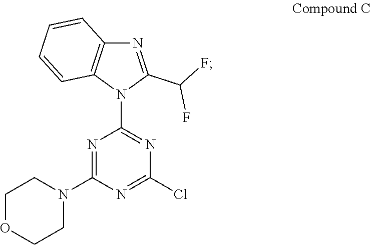 [SMILES: FC(F)c1nc2ccccc2n1-c1nc(Cl)nc(N2CCOCC2)n1]
is prepared by contacting Compound A: 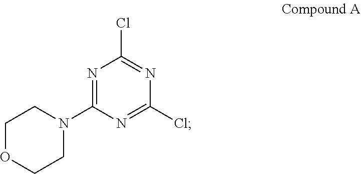 [SMILES: Clc1nc(Cl)nc(N2CCOCC2)n1]
with Compound B: 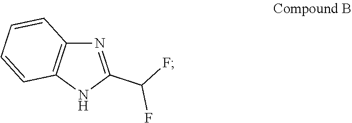 [SMILES: FC(F)c1nc2ccccc2[nH]1]
in the presence of a base and a solvent.

30. The process of claim 29, wherein the base is selected from sodium hydroxide, potassium carbonate, sodium carbonate, sodium bicarbonate, piperidine, 1,8-diazabicyclo[5.4.0]undec-7-ene, N,N-diisopropylethylamine, and triethylamine.

31. The process of claim 29 or 30, wherein the base is potassium carbonate.

32. The process of any one of claims 29-31, wherein the solvent is selected from water, ethyl acetate, dichloromethane, tetrahydrofuran, diethyl ether, dimethylformamide, dimethylsulfoxide, methanol, ethanol, acetone, acetonitrile, 1,4-dioxane, hexane, or methyl tert-butyl ether.

33. The process of any one of claims 29-32, wherein the solvent is acetone.

34. The process of any one of claims 29-33, wherein Compound A, Compound B, the base, and the solvent are stirred:
for no longer than 18 hours; and
at a temperature of between about 40° C. and about 50° C.

35. The process of any one of claims 29-34, comprising precipitating Compound C and isolating it by filtration.

36. The process of any one of claims 29-35, wherein the process provides Compound C in a synthetic yield of greater than about 80%.

37. The process of any one of claims 29-36, wherein the process provides Compound C in a synthetic yield of greater than about 90%.

38. A reaction mixture comprising 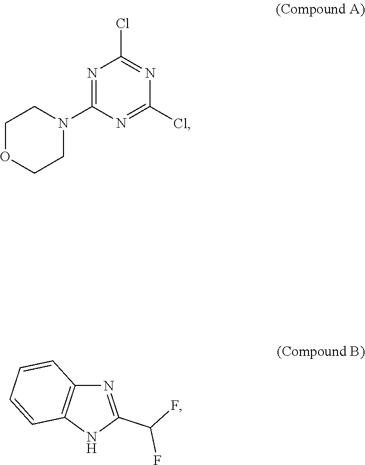 [SMILES: Clc1nc(Cl)nc(N2CCOCC2)n1 ; FC(F)c1nc2ccccc2[nH]1]
a base, and a solvent.

39. The reaction mixture of claim 38, wherein the base is potassium carbonate.

40. The reaction mixture of claim 38, wherein the solvent is acetone.

41. A reaction mixture comprising 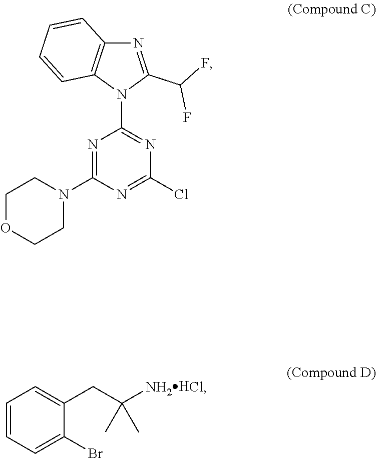
a base, and a solvent.

42. The reaction mixture of claim 41, wherein the base is potassium carbonate.

43. The reaction mixture of claim 41, wherein the solvent is 1,4-dioxane.

44. A reaction mixture comprising 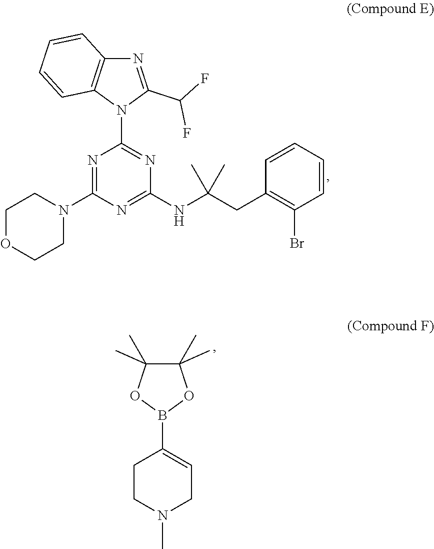
a base; a catalyst; and a solvent.

45. The reaction mixture of claim 44, wherein the base is potassium carbonate.

46. The reaction mixture of claim 44, wherein the catalyst is Pd(dppf)Cl₂.

47. The reaction mixture of claim 44, wherein the solvent is tetrahydrofuran.

48. A reaction mixture comprising 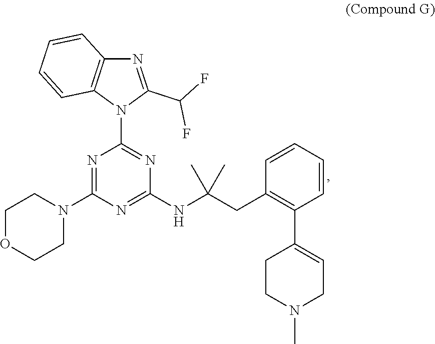 [SMILES: CN1CC=C(c2ccccc2CC(C)(C)Nc2nc(N3CCOCC3)nc(-n3c(C(F)F)nc4ccccc43)n2)CC1]
gaseous hydrogen; a catalyst; and a solvent.

49. The reaction mixture of claim 48, wherein the catalyst is Pd(OH)₂/C.

50. The reaction mixture of claim 48, wherein the solvent is 1,4-dioxane.

51. A compound that is Compound G: 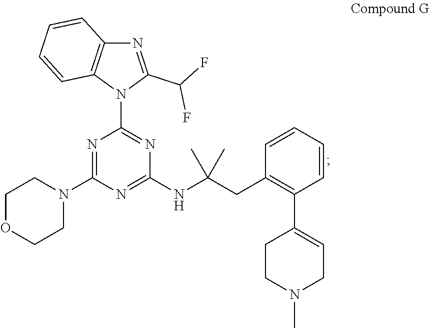 [SMILES: CN1CC=C(c2ccccc2CC(C)(C)Nc2nc(N3CCOCC3)nc(-n3c(C(F)F)nc4ccccc43)n2)CC1]
obtained by the process of claim 1.

52. A compound that is Compound 1: 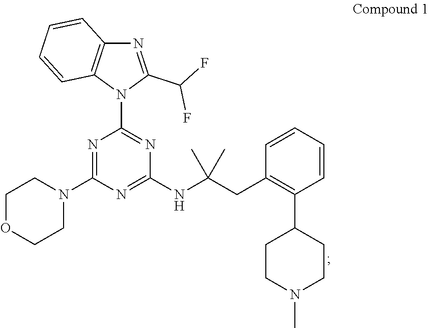 [SMILES: CN1CCC(c2ccccc2CC(C)(C)Nc2nc(N3CCOCC3)nc(-n3c(C(F)F)nc4ccccc43)n2)CC1]
obtained by the process of claim 12.

53. A compound that is Compound E: 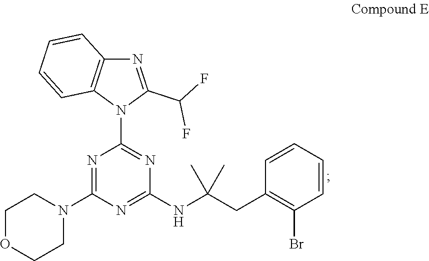 [SMILES: CC(C)(Cc1ccccc1Br)Nc1nc(N2CCOCC2)nc(-n2c(C(F)F)nc3ccccc32)n1]
obtained by the process of claim 20.

54. A compound that is Compound C: 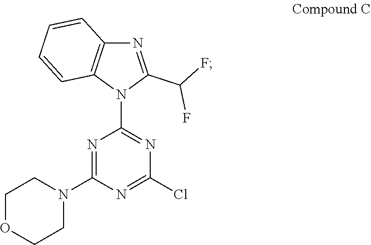 [SMILES: FC(F)c1nc2ccccc2n1-c1nc(Cl)nc(N2CCOCC2)n1]
obtained by the process of claim 29.

55. A compound that is Compound G: 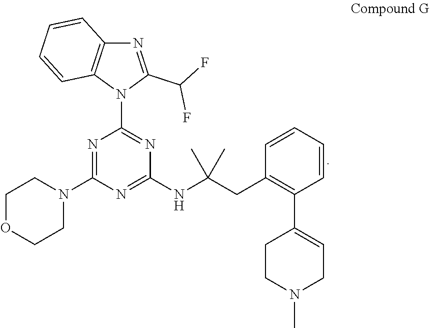 [SMILES: CN1CC=C(c2ccccc2CC(C)(C)Nc2nc(N3CCOCC3)nc(-n3c(C(F)F)nc4ccccc43)n2)CC1]
